(19) United States
(12) Patent Application Publication
(10) Pub. No.: US 2014/0276044 A1
(43) Pub. Date: Sep. 18, 2014

(54) DELIVERY CATHETER HAVING EXPANDABLE ELEMENTS

(71) Applicant: Volcano Corporation, San Diego, CA (US)

(72) Inventors: Jason Sproul, San Diego, CA (US); Timothy K. Glynn, San Marcos, CA (US)

(73) Assignee: VOLCANO CORPORATION, San Diego, CA (US)

(21) Appl. No.: 14/206,581

(22) Filed: Mar. 12, 2014

Related U.S. Application Data
(60) Provisional application No. 61/779,422, filed on Mar. 13, 2013.

(51) Int. Cl. A61M 5/00

(52) U.S. Cl. CPC A61M 5/007
USPC 600/435

(57) ABSTRACT

The invention is a delivery catheter, e.g., a guide catheter, having expandable elements proximate to the distal end of the catheter. Catheters of the invention are easier to place in proximity to an ostium, thereby increasing the efficiency of contrast delivery while reducing the risk of ischemia due to blocked blood supply. The invention additionally directs the flow of a fluid from the catheter, resulting in better performance with less fluid. For example, a catheter of the invention can be used to produce improved fluoroscopic images with less overall contrast. This improvement also decreases the length of time for a procedure, i.e., because of a need to re-contrast.

### RELATED APPLICATION

This application claims priority to, and the benefit of, U.S. Provisional Application No. 61/779.422, filed Mar. 13, 2013, which is incorporated by reference in its entirety.

### FIELD OF THE INVENTION

The invention relates to delivery catheters, e.g., guide catheters, having expandable elements to facilitate placement of the catheter and to direct the flow of a delivered fluid, e.g., a contrast agent.

### BACKGROUND

During intravascular procedures, devices used to perform medical procedures are typically visualized within the vasculature using contrast agents and real-time x-ray images. i.e., fluoroscopy. Often the contrast is introduced into the vasculature by placing a guide catheter through the heart and in proximity to an ostium leading to the coronary arteries, e.g., the left anterior descending coronary artery or the left circumflex coronary artery. In this configuration, contrast introduced via the guide catheter will be pushed through the coronary arteries with the natural pumping of the heart, thereby providing images of the arteries on the fluoroscope.

Placement of the guide catheter near an ostium is a delicate task, however. If the guide catheter is too far away, flushing is less complete and less effective. This can increase the need to add more contrast and increase the length of the flushes. The resultant increase in contrast loading increases the risk of ischemia in the tissues oxygenated by the arteries, while causing stress to the liver and kidneys. Alternatively, if the guide catheter completely blocks the ostium, the contrast will not circulate through the arteries and it will be very difficult to identify the vasculature through which the device is being inserted.

### SUMMARY

The invention is a delivery catheter, e.g., a guide catheter, having expandable elements in proximity to the distal end of the catheter. Catheters of the invention are easier to place in proximity to an ostium, thereby increasing the efficiency of contrast delivery while reducing the risk of ischemia due to blocked blood supply. The invention additionally directs the flow of a fluid from the catheter, resulting in better performance with less fluid. For example, a catheter of the invention can be used to produce improved fluoroscopic images with less overall contrast. This improvement reduces the risk of an allergic reaction to the contrast while decreasing the length of time for a procedure, i.e., because of a need to re-contrast.

### BRIEF DESCRIPTION OF THE DRAWINGS

FIG. 1 is a generalized depiction of a catheter having distal ports for use in delivering agents, e.g., contrast agents;

FIG. 2A depicts an embodiment of the invention having an inflatable member proximal to the distal end of the catheter;

FIG. 2B depicts an embodiment of the invention having an inflatable member proximal to the distal end of the catheter;

FIG. 3 illustrates deployment of a guide catheter of the invention at an ostium;

FIG. 4A illustrates an alternative use of a delivery catheter of the invention in a biological lumen;

FIG. 4B illustrates an alternative use of a delivery catheter of the invention in a biological lumen.

### DETAILED DESCRIPTION

The invention is a delivery catheter having expandable members that facilitate placement of the catheter while helping to direct the flow of a fluid delivered with the catheter. The design of the catheter can be used with any number of delivery catheters, however they are especially-suited to be used with guide catheters, such as the type used to deliver contrast agents during endovascular procedures. The expandable members can be balloons, shields, or flaps. In one embodiment, the expandable members are balloon-like elements that are inflated with an increase in pressure at the distal end of the catheter with delivery of a fluid.

Catheters having the described expandable members can be any type of catheter. Catheters include any elongated intraluminal device that comprises a tube for delivering the oxygenated particles within the body. In some instances, the catheters will perform additional functions, e.g., angioplasty, imaging, aspiration, pressure or flow measurement, etc. Many catheters for endovascular procedures are around 200 cm in total length and are administered via a cannula (introducer) that has been placed in an artery (e.g., the femoral, brachial, or radial artery). Catheters can be shorter, however, e.g., between 100 and 200 cm, or longer, e.g., between 200 and 400 cm. Often a vascular catheter is delivered along a guidewire. The catheter may have a lumen running the length of the catheter to accept a guidewire (over the rail) or only a portion of catheter, typically the distal tip 410, will have a guidewire lumen (rapid exchange).

In some instances, the catheters are placed with a guidewire. Access guidewires (generally “guidewires” herein) are known medical devices used in the vasculature or other anatomical passageway to act as a guide for other devices, e.g., a catheter. Typically, the guidewire is inserted into an artery or vein and guided through the vasculature under fluoroscopy (real time x-ray imaging) to the location of interest. In some procedures one or more devices are delivered over the guide wire to diagnose, image, or treat the condition. Guidewires typically have diameters of 0.010″ to 0.035″, with 0.014″ being the most common. Guidewires (and other intravascular objects) are also sized in units of French, each French being ⅓ of a mm or 0.013″. Guidewire lengths vary up to 400 cm, depending on the anatomy and work flow. The ends of the guidewire are denoted as distal (far from the user, i.e., inside the body) and proximal (near the user, i.e., outside the body). Often a guidewire has a flexible distal tip portion about 3 cm long and a slightly less flexible portion about 30 to 50 cm long leading up to the tip with the remainder of the guidewire being stiffer to assist in maneuvering the guidewire through tortuous vasculature, etc. The tip of a guidewire typically has a stop or a hook to prevent a guided device, e.g., a catheter from passing beyond the distal tip. In some embodiments, the tip can be deformed by a user to produce a desired shape.

In some instances, the catheter comprises a resilient inner coil, making it possible to shape the catheter and deliver it to targeted anatomy, e.g., the heart. Such catheters are generally known as guide catheters and are often used to deliver fluids, e.g., contrast agents to critical areas, or to deliver the catheter to tortuous locations, such as vasculature or the heart. Because the catheters are rather narrow, e.g., typically 8 French or less, it is also possible to guide a second catheter, for example, an imaging or therapeutic along the guide catheter to a desired location along the guide catheter.

An embodiment of a catheter system 10 for use with the invention is shown in FIG. 1. FIG. 1 is merely exemplary, as many other configurations of the catheter system 10 are possible to achieve the principles of the invention. The catheter system 10 includes a guide catheter 12 having a catheter body 14 with a proximal end 16 and a distal end 18. A luminal opening at the distal end 18 allows a fluid to be delivered to a patient. Catheter body 14 is flexible and defines a catheter axis 15, and may include multiple lumens, such as a guidewire lumen, an inflation lumen, and a fluid delivery lumen. Catheter 12 includes an inflatable balloon 19 (expandable member), proximal to the distal end 18, and a housing 29 adjacent proximal end 16. When inflated, balloon 19 assures than no more than a distance d of guide catheter can be inserted into an anatomical structure or it assures that at least a length d of guide catheter stays within a structure. Additional lumens may be provided for other treatments, such as irrigation, aspiration, perfusion, or delivery of a device, e.g., a stent.

In an embodiment, housing 29 includes a first connector 26 in communication with the guidewire lumen and a second connector 28 in fluid communication with the inflation lumen (not shown). In some embodiments, a separate inflation lumen is not necessary because the balloon 19 is inflated with pressure from the delivery lumen (see FIG. 2). In embodiments using an inflation lumen, the lumen extends between balloons 19 and second connector 28. Both first and second connectors 26, 28 may optionally comprise standard connectors, such as Luer-Loc™ connectors.

Housing 29 additionally provides a connection to a pump 38 coupled to a fluid source, e.g., saline or contrast. The pump 38 may be any pump suitable to deliver sufficient pressure to push the fluid through the delivery lumen to be administered via the distal opening. In some embodiments, the pump 38 will be designed to give an initial burst of pressure to inflate the balloon 19. The pump 38 may be a peristaltic pump, or the pump 38 may be a syringe. The reservoir 40 is any suitable reservoir to hold the fluid prior to delivery to the patient. The reservoir 40 may comprise pressure and temperature controls to maintain the fluid in optimum condition. In simple embodiments, both the pump 38 and the reservoir 40 take the form of a single syringe. In advanced embodiments, as shown in FIG. 1, the pump 38 and the particle reservoir 40 may be controlled by a controller 42 that is interfaced with patient monitoring equipment, e.g., a blood oxygen sensor, or a pressure sensor.

The controller 42 may include a processor, or is coupled to a processor, to control and/or record treatment. The processor will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of catheter system 10 and within processor 42.

While the guide catheter 12 of the catheter system 10 of FIG. 1 uses a separate inflation lumen to expand the balloon 19, the balloon can also be expanded using the pressure of the fluid to be delivered with the distal end 18 of the catheter, as shown in FIGS. 2A and 2B. In a first embodiment 100, shown in FIG. 2A, the distal end 18 does not have a restriction, thus fluid must be overpressurized to deliver the fluid and expand the balloon 19. In a second embodiment, shown in FIG. 2B, the distal end 18 comprises a restriction 120 causing the pressure of the delivered fluid to build up at the distal end 18, thereby expanding the balloon 19.

As shown in FIG. 3, the guide catheter having expandable members can be used to effectively and reliably place the distal tip 18 of the catheter inside an ostium 310 of an artery 330. This placement assures that the length d of the guide catheter inside the ostium is appropriate for the procedure. In some embodiments, the guide catheter will have markers distal to the balloon 19, allowing a surgeon to modify the length of the distance d as needed for the procedure. An additional benefit of the balloons is that fluids flushed from the catheter will be directed distally away from the tip. This feature is more readily appreciated on viewing FIG. 4, discussed below.

An alternative use of a catheter of the invention is shown in FIGS. 4A-4B. In this embodiment, the catheter 200 has been placed in a lumen 280, which may be an artery or vein on the order of the same size as the catheter 200. The catheter 200 includes a balloon 210, a catheter body 230, a distal tip 235, and a fluid delivery lumen 250. The balloon 210 has a proximal side (depicted as left in FIGS. 4A-4B) and a distal side (depicted as right in FIGS. 4A-4B). The balloon 210 expands radially when inflated with a fluid or a gas, e.g., as discussed above. The balloon 210 is constructed from a compliant material that can withstand high pressures.

The sequence of insertion, inflation, and fluid delivery is shown in FIGS. 4A-4B, respectively. As shown in FIG. 4A, prior to inflation, the balloon 210, positioned near the distal tip 235, is placed within a biological lumen 280. In an embodiment, the balloon 210 has helical folds to facilitate conversion between an expanded (inflated) configuration and a low profile configuration, needed for delivery and removal.

Once the catheter 200 is in place, the balloon 210 is inflated to obstruct the lumen, as shown in FIG. 2B. For example, the balloon 210 may be inflated with saline or another biocompatible fluid. Alternatively, the balloon 210 may be expanded with the delivery fluid, e.g., using the pressure of the fluid as described above with respect to FIGS. 2A and 2B. Once the balloon 210 is inflated, a fluid introduced via the distal end 235 can only travel to the distal side of balloon 210. When used with a contrast agent, the inflated balloon 210 will result in sharp line on the fluoroscope detailing the exact location of the catheter end. This method will also be useful if the catheter is inserted against the normal flow of fluid in the lumen, e.g., blood flow. Once the angioplasty procedure is complete, the balloon 210 is deflated, returning the catheter 200 to roughly the configuration in FIG. 4A. In this configuration, it is safe to remove the catheter 200 from the biological lumen 280.

The methods of the invention can also be used with various imaging catheters. For example, the imaging could be IVUS, OCT, or visible imaging. IVUS uses a catheter with an ultrasound probe attached at the distal end. Systems for IVUS are also discussed in U.S. Pat. No. 5,771,895, U.S. Pat. Pub. 2009/0284332, U.S. Pat. Pub. 2009/0195514, U.S. Pat. Pub. 2007/0232933, and U.S. Pat. Pub. 2005/0249391, the contents of each of which are incorporated herein by reference. OCT uses interferometric measurements to determine radial distances and tissue compositions. Systems for OCT imaging are discussed in U.S. Pat. No. 7,813,609 and US Patent Publication No. 20090043191, both of which are incorporated herein by reference in their entireties. Visible imaging can be accomplished using known digital imaging technology, for example, using a megapixel CCD arrays. The CCD array may be incorporated into the catheter, or the images can be collected with an optical fiber and directed onto a CCD outside of the catheter. In most instances, visible imaging will additionally require an illumination source, which can be provided using an optical fiber coupled to an illumination source, or an illumination source can be incorporated into the catheter, e.g., an LED. Direct imaging with other wavelengths, e.g., infrared, is also possible using known light sources and detection equipment. For example, a catheter could incorporate an infrared light source and an infrared photodiode array to produce infrared images of the interior of a lumen. The devices of the invention may include static imaging assemblies that do not move with respect to the catheter body, or the invention may include moving imaging assemblies. For example, the imaging assembly may be a phased array of ultrasonic transducers for IVUS imaging, or a collection of CCD arrays. An array of elements will typically cover a circumference of the catheter to provide a 360° view of the lumen. In other embodiments, the imaging assembly may rotate or translate using drive cables within the catheter body. Catheters having imaging assemblies that rotate and translate are known generally as “pull-back” catheters. The principles of pull-back OCT are described in detail in U.S. Pat. No. 7,813,609 and US Patent Publication No. 20090043191, both of which are incorporated herein by reference in their entireties. The mechanical components, including drive shafts, rotating interfaces, windows, and couplings, are similar between the various forms of pull-back imaging.

While the invention is described as delivering fluids, e.g., contrast, to the vasculature, it is understood that similar methods could be used to deliver fluids to a number of tissues that are accessible via the various lumen of the body, including, but not limited to vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, uterus and fallopian tubes, structures of the urinary tract including urinary collecting ducts, renal tubules, urethra, and bladder, and structures of the head and neck and pulmonary system including sinuses, parotid, trachea, bronchi, and lungs.

Additional embodiments of the invention including other combinations of oxygenated particle delivery, treatment and assessment will be evident to those of skill in the art in view of this disclosure and the claims below.

### INCORPORATION BY REFERENCE

References and citations to other documents, such as patents, patent applications, patent publications, journals, books, papers, and web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A delivery catheter comprising:
an elongated member having a proximal connector, a distal luminal opening, and a delivery lumen providing fluid communication between the proximal connector and the distal luminal opening; and
an expandable element disposed proximate to the distal luminal opening and adapted to expand to a diameter greater than the diameter of a distal tip of the elongated member.

2. The delivery catheter of claim 1, wherein the delivery catheter comprises a resilient member allowing a distal portion of the catheter to retain a predetermined shape.

3. The delivery catheter of claim 1, wherein the expandable element is selected from a balloon, a shield, and a tab.

4. The delivery catheter of claim 1, wherein the catheter is a guide catheter.

5. The delivery catheter of claim 1, wherein the distance between the distal tip and the expandable element is 5 cm or less.

6. The delivery catheter of claim 5, wherein the distance between the distal tip and the expandable element is 2 cm or less.

7. The delivery catheter of claim 1, wherein the elongated member additionally comprises an inflation lumen providing fluid communication between the expandable element and an inflation connector located at the proximal end of the elongated member.

8. The delivery catheter of claim 1, wherein the expandable element is fluidicly coupled to an inflation lumen, and the inflation lumen is in fluidic communication with the delivery lumen.

9. The delivery catheter of claim 8, wherein the expandable element is configured to be expanded with a pressurized fluid that is also delivered via the distal luminal opening.

10. The delivery catheter of claim 9, wherein the pressurized fluid comprises an angiographic contrast agent.

11. A method for delivering fluid to a vein or artery, comprising:
inserting a delivery catheter into a vein or artery, the delivery catheter having:
a proximal connector and a distal luminal opening and a delivery lumen providing fluid communication between the proximal connector and the distal luminal opening, and
an expandable element proximate to the distal luminal opening and in fluid communication with the delivery lumen; and
delivering a pressurized fluid through the delivery lumen thereby expanding the expandable element and delivering the pressurized fluid to the vein or artery via the distal luminal opening.

12. The method of claim 11, wherein the pressurized fluid comprises a contrast agent.
